(19) United States
(12) Patent Application Publication
(10) Pub. No.: US 2019/0388574 A1
(43) Pub. Date: Dec. 26, 2019

(54) STERILIZATION METHOD

(71) Applicant: PeroxyChem LLC, Philadelphia, PA (US)

(72) Inventors: Weidong AN, Williamsville, NY (US); Ricky MITTIGA, Tonawanda, NY (US); John ROVISON, Sanborn, NY (US)

(73) Assignee: PeroxyChem LLC, Philadelphia, PA (US)

(21) Appl. No.: 16/448,542

(22) Filed: Jun. 21, 2019

Related U.S. Application Data
(60) Provisional application No. 62/688,592, filed on Jun. 22, 2018.

(51) Int. Cl. A61L 2/20

(52) U.S. Cl. CPC A61L 2/20; A61L 2202/11

(57) ABSTRACT

Provided herein are methods and compositions for sterilizing a material with a composition comprising peracetic acid and a short chain organic acid stabilizer, for example, oxalic acid or malonic acid. The use of the short chain organic acid stabilizer results in a reduction of the amount of residue deposited on the heating surface used to vaporize the peracetic acid.

### CROSS REFERENCE TO RELATED APPLICATIONS

This application claims priority under 35 U.S.C. § 119(e)(1) from U.S. Provisional Application Ser. No. 62/688,592, filed Jun. 22, 2018, the contents of which are incorporated herein by reference.

### FIELD OF THE INVENTION

The present invention relates to peracetic acid-based compositions for vapor phase sterilization that results in reduced residue formation on the heating surface used to vaporize the peracetic acid.

### BACKGROUND OF THE INVENTION

Surfaces in the ambient environment are typically contaminated with microbes. Sterilization processes to eliminate such microbes are used in a wide variety of technologies including aseptic packaging, medical instrument handling, biocidal vector environmental remediation, food and beverage preparation and packaging, pharmaceutical manufacturing, wound dressing production, and electrical component fabrication. The choice of any one particular sterilization process depends on many factors, for example, the time required to kill or deactivate target microorganisms, the ability of the material to be sterilized to withstand exposure to high temperatures, elevated pressure, and moisture, and the associated costs. Ineffective processes can result in products that pose significant public health risks. There is a continuing need for sterilization processes and reagents that are effective, safe, and that do not adversely affect the material to be sterilized.

### SUMMARY OF THE INVENTION

Provided herein are methods of sterilizing a material. The method can include the steps of providing a sterilizing composition comprising (i) peracetic acid and (ii) a stabilizer selected from the group consisting of oxalic acid, mesoxalic acid, malonic acid, succinic acid, and tartronic acid; contacting the sterilizing composition with a heating surface to produce a peracetic acid vapor, introducing the peracetic acid vapor into a hot gaseous stream; and contacting the peracetic acid vapor in the gaseous stream with the material to be sterilized. The peracetic acid concentration can be from about 15 to about 17 weight percent of the sterilizing composition; and the stabilizer concentration can be about 0.05 and about 1.5 weight percent of the sterilizing composition. The stabilizer can be oxalic acid or malonic acid. The material can be a polymer, a metal, or glass. The polymer can be a polyethylene or an elastomer. The polyethylene can include ultra-high molecular weight polyethylene (UHMWPE), high density polyethylene (HDPE), medium density polyethylene (MDPE), low density polyethylene (LDPE) and polyethylene terephthalate (PET). In some embodiments, the polymer can be polystyrene, polycarbonate, polylactylate, or polylactone. In some embodiments, elastomer can be polytetrafluoroethylene (PTFE), a perfluoroethoxy alkane (PFA), latex rubber, or neoprene. The hot gaseous stream can be sterile air. In some embodiments, the hot gaseous stream can be nitrogen, carbon dioxide, a noble gas or a mixture thereof. The hot gaseous stream can be heated to a temperature above about 250° C. prior to the introduction of the peracetic acid. The hot gaseous stream can be heated to a temperature above about 250° C. and then cooled to a temperature of between about 80° C. and about 120° C. prior to the introduction of the peracetic acid. The temperature of the hot gaseous stream is at least about 5° C. higher than the dew point of peracetic acid. The contact between the peracetic acid vapor and the material to be sterilized can be maintained for about 10 seconds. The PAA is an aqueous equilibrium composition having a PAA:hydrogen peroxide:acetic acid weight ratio can include 12-18:21-24:5-20; 15:6:10; 15:10:36; 5:23:10; 21-23:6-12:21-35; and 3.5:10:15.

### DETAILED DESCRIPTION OF THE PREFERRED EMBODIMENT

This description of preferred embodiments is intended to be read in connection with the accompanying drawings, which are to be considered part of the entire written description of this invention. The drawing FIGURES are not necessarily to scale and certain features of the invention may be shown exaggerated in scale or in somewhat schematic form in the interest of clarity and conciseness. In the description, relative terms such as “horizontal,” “vertical,” “up,” “down,” “top” and “bottom” as well as derivatives thereof (e.g., “horizontally,” “downwardly,” “upwardly,” etc.) should be construed to refer to the orientation as then described or as shown in the drawing FIGURE under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. The term “operatively connected” is such an attachment, coupling or connection that allows the pertinent structures to operate as intended by virtue of that relationship. When only a single machine is illustrated, the term “machine” shall also be taken to include any collection of machines that individually or jointly execute a set (or multiple sets) of instructions to perform any one or more of the methodologies discussed herein. In the claims, means-plus-function clauses, if used, are intended to cover the structures described, suggested, or rendered obvious by the written description or drawings for performing the recited function, including not only structural equivalents but also equivalent structures.

The present invention is directed to methods and compositions for peracetic acid vapor phase sterilization of a surface. A true vapor is a state in which the peracetic acid is substantially entirely in the gaseous form. This is in contrast to a mist or fog, both of which contain a significant proportion of liquid droplets suspended in the air. Such a “dry vapor” system resulted in effective biocidal activity without the formation of water droplets on the treated surface. A dry vapor is typically produced by contacting a peracetic acid solution directly with a heating surface at a temperature that results in vaporization of the peracetic acid.

Peracetic acid (PAA) solutions are typically formulated to include a stabilizer, for example phosphonic acid or phosphonic acid derivatives such as 1-hydroxyethylidene-1,1,-diphosphonic acid (Dequest™2010) to prolong shelflife. Over time, repeated contact of standard stabilized peracetic acid solutions with the heating surface results in undesirable deposition of residue on the heating surface. Residue buildup can decrease the heat transfer from the heating element and thus decrease vaporization efficiency and sterilization effectiveness. Residue buildup is generally a function of the length of time that the peracetic acid vapor remains in contact with the heating surface. Residue buildup can be exacerbated in sterilization equipment in which the heating element has a lower thermal driving force and thus takes longer to achieve vaporization temperature. The more prolonged contact time generally does not result in flash vaporization, which, without wishing to be bound by theory, may contribute to increased residue buildup. The residue is generally composed of the stabilizer and/or breakdown products of the stabilizer. Removal of the residue from the heating surface requires a shutdown and disassembly of the sterilizing apparatus and is thus is time-consuming and costly.

The Applicant has found that the combination of peracetic acid with a short chain organic acid, for example, oxalic acid or malonic acid, resulted in reduced residue deposition on the heating surface used to vaporize the peracetic acid. The reduction in residue deposition is useful under sterilizing conditions in which evaporation takes place more slowly and under lower temperatures. Surprisingly, such short chain organic acids effectively stabilized peracetic acid solutions. And, compositions comprising peracetic acid and a short chain organic acid, for example, oxalic acid or malonic acid were effective sterilizing agents.

The compositions disclosed herein include peracetic acid. Peracetic acid is typically employed in the form of an aqueous equilibrium mixture of acetic acid, hydrogen peroxide and peracetic acid. The weight ratios of these components can vary. Peracetic acid solutions can be identified by the concentration of peracetic acid and hydrogen peroxide. Commercially available peracetic acid solutions have typical formulations containing 2-35% peracetic acid and 5-30% hydrogen peroxide, with the remainder being acetic acid and water. Exemplary peracetic acid solutions can include 15% peracetic acid with 10% hydrogen peroxide; 22% peracetic acid with 10% hydrogen peroxide; 35% peracetic acid with 7% hydrogen peroxide; 15% peracetic acid with 3% hydrogen peroxide; 22% peracetic acid with 4% hydrogen peroxide. Exemplary peracetic acid solutions which can be used include those having weight ratios of peracetic acid:hydrogen peroxide:acetic acid from 5:23:10; 12-18:21-24:5-20; 15:6:10; 15:10:36; 15:10:35; 5:23:10; 21-23:6-12:21-35; and 35:10:15.

The stabilizer can be a short chain organic acid, that is, an organic acid having 5, 4 or fewer single bonded carbon atoms. Useful short chain organic acids can have 4 single bonded carbon atoms; 3 single bonded carbon atoms; or 2 single bonded carbon atoms. Useful short chain organic acids can include 2 or fewer dicarboxylic acids. In some embodiments the short chain organic acid is unbranched. A short chain organic acid can be, for example, oxalic acid, mesoxalic acid, malonic acid, succinic acid, and tartronic acid or a combination of any of oxalic acid, mesoxalic acid, malonic acid, succinic acid, and tartronic acid. In some embodiments, the stabilizer is oxalic acid. In some embodiments, the stabilizer is malonic acid. The inventors have found surprisingly that short chain organic acids effectively stabilized peracetic acid solutions.

The short chain organic acid is combined with the peracetic acid in an amount sufficient to stabilize the peracetic acid for a period of at least six months. The peracetic acid solution will generally retain at least about 80% of the original percent of active oxygen after storage at room temperature for a period of at least about 180 days.

The stabilizer, that is, the short chain organic acid, can be added directly to any of the peracetic acid aqueous equilibrium solutions described above to produce a sterilizing composition. The concentration of the short chain organic acid in the sterilizing composition can range from about 0.1% to about 2.0% by weight based on the total weight of the composition. Thus the concentration of the short chain organic acid can be about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, or about 2.0%.

The sterilizing composition can include or exclude a sequestrant such as dipicolinic acid. The sterilizing composition can further include or exclude a mineral acid catalyst, for example, sulfuric acid, nitric acid, or phosphoric acid. The sterilizing composition can also include or exclude a surfactant, for example, an anionic laurylate or a sorbitan as well as their respective esters, i.e. polyethylene sorbitan monolaurylates; and short chain fatty esters (C6-C12) forming mixed peracids in solution. In some embodiments, the sterilizing composition can include or exclude one or more additional oxidants selected from the group consisting of chloroperbenzoic acid, perheptanoic acid, peroctanoic acid, perdecanoic acid, performic acid, percitric acid, perglycolic acid, perlactic acid and perbenzoic acid.

The sterilizing composition can be diluted prior to use, that is, prior to contacting the composition with a heating element. The sterilizing composition can be diluted by the addition of high quality water, for example deionized water with ≥2 MOhm resistivity or ≥0.5 μSiemens conductivity, to a working concentration of less than about 100,000 parts per million (ppm) of peracetic acid. Thus, the working concentration of the peracetic acid in the composition can range from about 1 ppm to about 100,000 ppm. Thus the concentration of the peracetic acid can be about 1 ppm, about 2 ppm, about 3 ppm, about 4 ppm, about 5 ppm, about 6 ppm, about 7 ppm, about 8 ppm, about 9 ppm, about 10 ppm, about 12 ppm, about 15 ppm, about 18 ppm, about 20 ppm, about 25 ppm, about 30 ppm, about 35 ppm, about 40 ppm, about 45 ppm, about 50 ppm, about 60 ppm, about 75 ppm, about 100 ppm, about 125 ppm, about 150 ppm, about 200 ppm, about 250 ppm, about 300 ppm, about 350 ppm, about 400 ppm, about 450 ppm, about 500 ppm, about 1000 ppm, about 1500 ppm, about 2000 ppm, about 2200 ppm, about 2500 ppm, about 2900 ppm, about 3000 ppm, about 3500 ppm, about 4000 ppm, about 4500 ppm, about 5000 ppm, about 6000 ppm, about 7500 ppm, about 8,000 ppm, about 10,000 ppm, about 12,000 ppm, about 14,000 ppm, about 15,000 ppm, about 16,000 ppm, about 18,000 ppm, about 20,000 ppm, about 22,000 ppm, about 24,000 ppm, about 25,000 ppm, about 26,000 ppm, about 28,000 ppm, about 30,000 ppm, about 32,000 ppm, about 34,000 ppm, about 35,000 ppm, about 36,000 ppm, about 38,000 ppm, about 40,000 ppm, about 50,000 ppm, about 60,000 ppm, about 70,000 ppm, about 80,000 ppm, about 90,000 ppm, or about 100,000 ppm.

The working concentration of the small organic acid can range from about 500 ppm to about 3000 ppm. Thus the concentration can be about 500 ppm, about 600 ppm, about 700 ppm, about 800 ppm, about 900 ppm, about a 1000 ppm, about 1200 ppm, about 1400 ppm, about 1500 ppm, about 1600 ppm, about 1800 ppm, about 2000 ppm, about 2200 ppm, about 2400 ppm, about 2500 ppm, about 2600 ppm, about 2800 ppm, or about 3000 ppm.

The diluted sterilizing composition is contacted with a heating surface to produce a peracetic acid vapor. The temperature of the heating surface should be sufficient to vaporize the peracetic acid. The temperature of the heating surface can vary, but in general, should be high enough to produce a vapor rather than a fog or mist. But the temperature should not be so high as to either decompose the peracetic acid or to result in the Leidenfrost effect in which droplets become suspended in insulating vapor and hover over the surface to be sterilized. Useful heating surface temperatures can range from about 120° C. to about 220° C. The configuration of the heating surface can vary. A heating surface can be, for example, a flat plate, a steam heating coil or spiral wedge with internal steam or electrical heating elements and/or an indirectly heated chamber with external steam, electrical or radiant heat.

The vaporized peracetic acid can be introduced into the hot gaseous stream using a variety of methods, for example, by direct injection. The heated gas stream is typically sterile air, although other gases such as superheated steam (without droplets) nitrogen, carbon dioxide, or inert noble gas carriers may also be employed. Such gas stream is typically heated to a temperature of at least about 300° C., preferably to a minimal temperature of about 250° C., and can be in excess of 350° C. providing it can be cooled sufficiently for application. It then is typically cooled to between about 80° C. and about 120° C. prior to the introduction of the vaporized peracetic acid. The heated gas stream at the point of PAA introduction should have a temperature of at least 5° C. higher than the dew point of PAA (ca. 46.5-49.9° C.); i.e., of at least about 55° C., to ensure that the peracetic acid is maintained as a vapor rather than a fog or mist. In general, the heated gas stream is less than 100% saturated. In some embodiments the heated gaseous stream is between about 75 and 85% saturated.

The gaseous PAA vapor is then contacted with the material to be sterilized for a time sufficient to kill the contaminants of concern. This time will vary according to many factors such as the concentration of the PAA vapor employed; the nature of the material surface to be sterilized; the contaminants to be sterilized; the contaminant concentrations; and the target Log₁₀ reduction efficacy level; and the like. Typically, such contact will be maintained at the level of a few seconds for aseptic packaging applications.

The contact time between the peracetic acid vapor and the material to be sterilized can vary depending upon the nature of the material and the particular microorganism being targeted. The contact time between the compositions and the substrate can range from a few seconds to more than one hour. Exemplary contact times include about 1 second, about 2 seconds, about 3 seconds, about 4 seconds, about 5 seconds, about 6 seconds, about 7 seconds, about 8 seconds, about 9 seconds, about 10 seconds, about 15 seconds, about 20 seconds, about 25 seconds, about 30 seconds, about 40 seconds, about 45 seconds, about 50 seconds, about 60 seconds, about 90 seconds, about 120 seconds, about 3 minutes, about 5 minutes, about 8 minutes, about 10 minutes, about 15 minutes, about 30 minutes, about 45 minutes, or about 60 minutes.

In general, a reduction of microbial contamination can be assayed by determining the level of viable microbes on the treated material. In some embodiments, a reduction of microbial contamination can be a reduction of about 50%, about 80% about 90%, about 95%, about 99% or about 99.9% of the contamination of the treated food product compared to an untreated control substrate. Alternatively, or in addition, the reduction can be specified as a Log₁₀ reduction. Thus in some embodiments, a reduction of microbial contamination can be a 1, 2, 3, 4, 5, 6, or 7 Log reduction relative to an untreated control substrate. Levels of microbial contamination can be determined, for example, by standard cultural methods involving microbial outgrowth, nucleic acid amplification techniques such as polymerase chain reaction, and immunoassays.

A wide variety of materials may be sterilized using the methods disclosed herein. The material can comprise a polymer, a metal, or glass. The polymer can be polyethylene or an elastomer. The polyethylene can be ultra-high molecular weight polyethylene (UHMWPE), high density polyethylene (HDPE), medium density polyethylene (MDPE), low density polyethylene (LDPE) or polyethylene terephthalate (PET). The polymer can be, for example, polystyrene, polycarbonate, polylactylate, or polylactone. An elastomer can be, for example, polytetrafluoroethylene (PTFE), a perfluoroethoxy alkane (PFA), latex rubber, or neoprene. In some embodiments, the material can include food or beverage packaging, for example, PET bottles and containers.

The method disclosed herein can be used to sterilize materials contaminated with any of a wide variety of microorganisms. Exemplary species include microbes typically controlled by peracetic acid in liquid form. These include bacteria and spores of the genus Bacillus using B. cereus, B. thuringiensis and B. atrophaeus as surrogates for more pathogenic species such as Clostridium botulinum as well as Staphylococcus, Enterococcus, Salmonella, Campylobacter, Pseudomonas, Candida, Rhizopus, Mucor, Influenza, or Bacilli. The compositions can be applied to both aerobic microorganisms and anaerobic microorganisms, for example, gram positive bacteria such as Staphylococcus aureus, Bacillus species (sp.) such as Bacillus subtilis, Clostridia sp.; gram negative bacteria, e.g., Escherichia coli, Pseudomonas sp. such as Pseudomonas aeruginosa and Pseudomonas fluorescens, Klebsiella pneumoniae, Legionella pneumophila, Enterobacter sp. such as Enterobacter aerogenes, Serratia sp. such as Serratia marcesens. Other exemplary bacteria can include Paenibacillus chibensis, Paenibacillus ebina, Paenibacillus flavisporus and Chaetomium globosum. The methods disclosed herein can also be used to sterilize materials contaminated with yeasts, e.g., Saccharomyces cerevisiae, Candida albicans; molds, e.g., Cephalosporium acremonium, Penicillium notatum, Aureobasidium pullulans; filamentous fungi, e.g., Aspergillus niger, Cladosporium resinae; algae, e.g., Chlorella vulgaris, Euglena gracilis, Selenastrum capricornutum; and other analogous microorganisms, e.g., phytoplankton and protozoa; viruses e.g., hepatitis virus, and enteroviruses such poliovirus, echo virus, coxsackie virus, norovirus, SARS, and JC virus.

### EXAMPLES

### Example 1

Chemicals.

Malonic acid (99%, CAS#141-82-2) and oxalic acid, anhydrous (98%, CAS#144-62-7) was purchased from VWR. Peracetic acid was used as an equilibrium peracetic acid solution having a weight ratio 15:10:35 of peracetic acid:hydrogen peroxide:acetic acid.

### Example 2

The effect of oxalic acid and malonic acid on PAA stability was evaluated. Varying amounts of oxalic acid (0.4%, 0.5%, or 1.0%) or malonic acid (0.5% or 1.0%) were added to concentrated PAA solutions and the resulting compositions were stored at room temperature. At intervals, aliquots of the compositions were analyzed to determine the peracetic acid (PAA); hydrogen peroxide (H₂O₂); and acetic acid (AA) contents (in percent by weight) and the Active Oxygen Recovery percentage (AO Rec). Total available active oxygen (“AO”), that is, the summation of active oxygen across the total number of peroxygen containing moieties, was calculated according to the formula: AO=Σnₓ_{^{−}}, wherein n=the amount active oxygen for each compound in the solution and x is the number of active oxygen containing components. The percent of active oxygen for a given compound can be determined by MW 02/MW compound×100%. Peracetic acid contains 16/76×100%, which is 21% of active oxygen. Hydrogen peroxide contains 16/34×100%, which is 47% of active oxygen. Thus, the total amount AO can be calculated as: [peracetic acid wt %]×0.21+[hydrogen peroxide wt %]×0.47.

As shown in Tables 1-5, both oxalic acid and malonic acid stabilized the peracetic acid for periods of more than 3 or 6 months.

TABLE 1
Peracetic acid stability in the presence of 0.4% oxalic acid

| Days after | Peracetic | Hydrogen | Acetic | Active |
|---|---|---|---|---|
| addition | acid % | peroxide % | acid % | oxygen % |
| 0.0 | 16.04 | 10.09 | 34.09 | 8.12 |
| 21.98 | 15.87 | 10.16 | 33.73 | 8.12 |
| 36.92 | 15.81 | 9.73 | 34.25 | 7.90 |
| 55.91 | 15.35 | 9.75 | 34.51 | 7.82 |

TABLE 2
Peracetic acid stability in the presence of 0.5% oxalic acid

| Days after | Peracetic | Hydrogen | Acetic | Active |
|---|---|---|---|---|
| addition | acid % | peroxide % | acid % | oxygen % |
| 0.0 | 16.31 | 10.44 | 31.81 | 8.34 |
| 124.00 | 16.24 | 10.11 | 32.66 | 8.17 |
| 189.00 | 15.84 | 10.16 | 33.90 | 8.11 |

TABLE 3
Peracetic acid stability in the presence of 1.0% oxalic acid

| Days after | Peracetic | Hydrogen | Acetic | Active |
|---|---|---|---|---|
| addition | acid % | peroxide % | acid % | oxygen % |
| 0.0 | 16.30 | 10.38 | 32.33 | 8.31 |
| 124.00 | 16.11 | 10.05 | 33.26 | 8.12 |
| 189.00 | 15.67 | 10.11 | 33.11 | 8.05 |

TABLE 4
Peracetic acid stability in the presence of 0.5% malonic acid

| Days after | Peracetic | Hydrogen | Acetic | Active |
|---|---|---|---|---|
| addition | acid % | peroxide % | acid % | oxygen % |
| 0.0 | 16.21 | 10.27 | 34.29 | 8.24 |
| 26.99 | 16.45 | 10.01 | 34.21 | 8.17 |
| 43.03 | 16.15 | 9.90 | 33.31 | 8.05 |
| 76.04 | 15.87 | 9.84 | 33.66 | 7.97 |
| 105.05 | 15.66 | 9.81 | 33.21 | 7.91 |
| 147.99 | 14.95 | 9.52 | 35.16 | 7.62 |
| 182.01 | 14.77 | 9.37 | 36.10 | 7.51 |
| 226.10 | 14.41 | 9.16 | 35.78 | 7.34 |

TABLE 5
Peracetic acid stability in the presence of 1.0% malonic acid

| Days after | Peracetic | Hydrogen | Acetic | Active |
|---|---|---|---|---|
| addition | acid % | peroxide % | acid % | oxygen % |
| 0.0 | 16.38 | 10.19 | 33.98 | 8.24 |
| 26.99 | 16.06 | 9.96 | 34.17 | 8.06 |
| 43.03 | 15.83 | 9.77 | 33.50 | 7.92 |
| 76.04 | 15.44 | 9.65 | 34.04 | 7.79 |
| 105.05 | 15.18 | 9.58 | 33.45 | 7.70 |
| 147.99 | 14.47 | 9.26 | 36.55 | 7.40 |
| 182.01 | 13.99 | 9.01 | 36.59 | 7.18 |
| 226.10 | 13.44 | 8.73 | 37.42 | 6.94 |

### Example 3

The amount of residue buildup produced by vaporization of PAA solutions in the presence of various stabilizers was assayed. The solutions included:

TABLE 6
Peracetic acid/stabilizer solutions

| Sample | Peracetic acid (ppm) | Stabilizer (ppm) |
|---|---|---|
| A1 (control) | 24,000 | 1200 ppm citric acid |
| A2 (control | 24,000 | 2400 ppm citric acid |
| B1 | 30,000 | 1000 ppm oxalic acid |
| B2 | 30,000 | 1000 ppm oxalic acid |
| B3 | 30,000 | 1875 ppm oxalic acid |
| C1 | 30,000 ppm | 940 ppm malonic acid |

A portion of each solution in Table 6 was aliquoted into a separate 4 L bottle. A pre-weighed stainless-steel pan was used for residue collection. Prior to collection, the pan was heated to 180-185° C. using a Corning Stirrer/Hotplate. Once the pan had reached temperature, the solution to be tested was vaporized by dropwise addition to the heated pan using a Chrome Tech Series II lab pump at a flow rate of 6.5 mL/min. The total volume added over time was monitored with a stopwatch. After a fixed time, the hotplate was turned off and the pan was allowed to cool to room temperature. The cooled pan was reweighed to determine the amount of residue present. The results of this analysis are shown in Table 7.

TABLE 7
Residue formation by Peracetic acid/stabilizer solutions

| | Residue | Volume of solution | mg/mL |
|---|---|---|---|
| Sample | (mg) | (mL) | of residue |
| A1 (control) | 0.1 | 187 | 0.001 |
| A2 (control) | 0.1 | 158 | 0.001 |
| B1 | 0.0 | 2275.4 | 0.000 |
| B2 | 5.0 | 2048.3 | 0.002 |
| B3 | 0.0 | 1907.9 | 0.000 |
| C1 | 0.0 | 2249.7 | 0.000 |

As shown in Table 7, the citric acid stabilized PAA control samples produced little or no residue. Both oxalic acid-stabilized PAA and malonic acid stabilized PAA also produced little or no residue even though substantially larger volumes of oxalic acid-stabilized PAA and malonic acid stabilized PAA were used compared to the citric acid-stabilized PAA control.

### Example 4

The antimicrobial efficacy of oxalic acid-stabilized PAA was compared with the antimicrobial efficacy of citric acid-stabilized PAA. The PAA stabilized solutions were prepared as described above. The stabilizer concentration for all solutions was 0.4%. B. cereus 14579 and B. atrophaeus 9372 spores were spot inoculated at the bottom of 500 mL polyethylene terephthalate (PET) bottles to provide at least 6 Log₁₀/bottle of microbes. The inoculated bottles were dried overnight in a biosafety cabinet. The inoculated bottles were then exposed to a five second paper TAA treatment. The bottles were neutralized immediately following the PAA treatment by the addition of 100 mL Letheen Broth with 0.5% sodium thiosulfate using aseptic technique. The bottles were capped and shaken to ensure that the vapor that had condensed on the sides was mixed with the neutralizer. The bottles were then sonicated for 5 minutes, and vortex mixed for 30 seconds, followed by serial dilution and plating on Petrifilm and TSA filter plate. Filter plates and Petrifilm were incubated at 35° C. for about 48 hours before counting.

The results of this analysis are shown in Table 8.

TABLE 8
Antimicrobial efficacy of oxalic acid stabilized PAA

| | PAA in diluted | | Inoculation | Efficacy |
|---|---|---|---|---|
| | solution | | control | (Log₁₀ |
| Stabilizer | (wt %) | Spores | (Log₁₀) | reduction) |
| Citric acid | 1.9 | B. atrophaeus | 6.4 ± 0.2 | Total kill |
| | | 9372 | | |
| Oxalic acid | 2.0 | B. atrophaeus | 6.4 ± 0.2 | Total kill |
| | | 9372 | | |
| Citric acid | 2.9 | B. cereus | 7.6 ± 0.1 | 5.8 ± 1.2 |
| | | 14579 | | |
| Oxalic acid | 3.0 | B. cereus | 7.6 ± 0.1 | 6.2 ± 0.6 |
| | | 14579 | | |

What is claimed is:

1. A method of sterilizing a material, the method comprising:
a) providing a sterilizing composition comprising (i) peracetic acid and (ii) a stabilizer selected from the group consisting of oxalic acid, mesoxalic acid, malonic acid, succinic acid, and tartronic acid;
b) contacting the sterilizing composition with a heating surface to produce a peracetic acid vapor,
c) introducing the peracetic acid vapor into a hot gaseous stream; and
d) contacting the peracetic acid vapor in the gaseous stream with the material to be sterilized.

2. The method of claim 1, wherein the peracetic acid concentration is from about 15 to about 17 weight percent of the sterilizing composition; and the stabilizer concentration is about 0.05 and about 1.5 weight percent of the sterilizing composition.

3. The method of claim 1, wherein the stabilizer concentration is between about 0.25 to about 1.25 weight percent of the sterilizing composition.

4. The method of claim 1, wherein the stabilizer concentration is between about 0.5 to about 1.0 weight percent of the sterilizing composition.

5. The method of claim 1, where in the stabilizer is oxalic acid or malonic acid.

6. The method of claim 1, wherein the stabilizer is oxalic acid.

7. The method of claim 1, wherein the stabilizer is malonic acid.

8. The method of claim 1, where in the peracetic acid concentration is less than 100,000 ppm.

9. The method of claim 1, wherein the peracetic acid concentration is less than 60,000 ppm.

10. The method of claim 1, wherein the material comprises a polymer, a metal, or glass.

11. The method of claim 10, wherein the polymer is a polyethylene or an elastomer.

12. The method of claim 11, wherein the polyethylene is selected from the group consisting of ultra-high molecular weight polyethylene (UHMWPE), high density polyethylene (HDPE), medium density polyethylene (MDPE), low density polyethylene (LDPE) and polyethylene terephthalate (PET).

13. The method of claim 1, wherein the polymer is selected from the group consisting of polystyrene, polycarbonate, polylactylate, or polylactone.

14. The method of claim 1, where in the elastomer is selected from the group consisting of polytetrafluoroethylene (PTFE), a perfluoroethoxy alkane (PFA), latex rubber, or neoprene.

15. The method of claim 1 wherein the hot gaseous stream comprises sterile air.

16. The method of claim 1 wherein the hot gaseous stream comprises nitrogen, carbon dioxide, a noble gas or a mixture thereof.

17. The method of claim 1 wherein the hot gaseous stream is heated to a temperature above about 250° C. prior to the introduction of the peracetic acid.

18. The method of claim 1 wherein the hot gaseous stream is heated to a temperature above about 250° C. and then cooled to a temperature of between about 80° C. and about 120° C. prior to the introduction of the peracetic acid.

19. The method of claim 1 wherein the temperature of the hot gaseous stream is at least about 5° C. higher than the dew point of peracetic acid.

20. The method of claim 1 wherein the contact between the peracetic acid vapor and the material to be sterilized is maintained for about 10 seconds.

21. The method of claim 1 wherein the contact between the peracetic acid vapor and the material to be sterilized is maintained for about 5 seconds.

22. The method of claim 1 wherein the sterilizing composition further comprises one or more oxidants selected from the group consisting of chloroperbenzoic acid, perheptanoic acid, peroctanoic acid, perdecanoic acid, performic acid, percitric acid, perglycolic acid, perlactic acid and perbenzoic acid.

23. The method of claim 1 wherein the PAA is an aqueous equilibrium composition having a PAA:hydrogen peroxide:acetic acid weight ratio selected from the group consisting of 12-18:21-24:5-20; 15:6:10; 15:10:36; 5:23:10; 21-23:6-12:21-35; and 3.5:10:15.
